(19) United States
(12) Patent Application Publication
(10) Pub. No.: US 2017/0348477 A1
(43) Pub. Date: Dec. 7, 2017

(54) CONTAINER FOR ENCLOSING A MEDICAL DEVICE

(71) Applicants: SHARON TOMLINSON, POST FALLS, ID (US); MARK FORD, SPOKANE, WA (US)

(72) Inventors: SHARON TOMLINSON, POST FALLS, ID (US); MARK FORD, SPOKANE, WA (US)

(21) Appl. No.: 15/174,557

(22) Filed: Jun. 6, 2016

(51) Int. Cl. A61M 5/00; B65D 65/14; B65D 75/22; B65D 75/58

(52) U.S. Cl. CPC A61M 5/002; B65D 75/5827; B65D 65/14; B65D 75/22

(57) ABSTRACT

A container for enclosing a medical device is disclosed, and in one form includes a flexible sheet bearing an adhesive layer which is deposited on a portion thereof, and wherein the flexible sheet can be folded or closed in a manner so as to create a cavity which receives a medical device therein, and which further allows a clinician to determine if the medical device has been accessed or tampered with by an unauthorized individual.

### TECHNICAL FIELD

The present invention relates to a container for enclosing a medical device, and more specifically, and in a first form of the invention, to a single-use flexible container which impedes, or substantially prevents unauthorized access and potential tampering with the medical device which is enclosed as a patient undergoes a clinician directed, and supervised medical treatment, and/or inhibits dust, debris and other biological contamination from coming into contact with the medical device during a treatment regimen.

### BACKGROUND OF THE INVENTION

Those skilled in the art will recognize that assorted clinician directed medical procedures include, as a part of a given treatment plan, the introduction of various medicines, and fluids by means of vascular infusion. In order to accomplish this treatment, assorted vascular infusion systems have been developed, over time. It should be understood that an infusion system is the process by which an infusion device, and associated disposables (Vascular Access Devices (VAD's)), are used to deliver fluids or drugs, in solution, to a patient by intravenous subcutaneous, epidural, or enternal routes. Such vascular infusion systems for the delivery of these fluids or other medicines may include monitors, controllers, syringe driver/pumps, general-purpose/volumetric pumps, PCA pumps, ambulatory pumps, and the like. Still further these vascular infusion systems also cooperate with tubing of various lengths and diameters; filters; taps; anti-siphon and free-flow prevention valves; clamps; and catheters of assorted designs, and which are used and matched to the infusion system which has been selected by skilled medical personnel.

For example, a peripherally inserted central catheter [PICC or PIC catheter] is a form of vascular access device that can be used for a prolonged period of time such as for chemotherapy regimens, extended antibiotic treatment or total parenteral nutrition. Such catheters can also be used for the administration of substance that should not be done peripherally. This particular catheter enters the body through the skin at a peripheral site, and extends to the superior vena cava, and stays in place for days or even sometimes weeks. PIC lines or catheters often are coupled in fluid receiving relation relative to a PICC hub and which allows a clinician to gain access to the PIC line or catheter to introduce various fluids at given time intervals during the treatment regimen which has been selected. Other designs of VAD are, of course, available.

As of late, many clinicians have noted an increase in the number of incidents within a clinical or treatment setting where a patient, or individuals acquainted with a patient have gained improper and unauthorized access to a Vascular Access Device for purposes of trying to introduce or inject unauthorized substances through the VAD into the vascular system of the patient being treated. Sometimes the motivation underlying the unauthorized access is due, in part, to the mental state of the patient undergoing treatment. On other occasions, the motivation for the unauthorized access is for other nefarious purposes. Apart from the aforementioned patient population, other patient groups such as home health patients; pediatric; hemodialysis; psychiatric; and patients in extended care facilities could benefit from a product which would prevent access or contamination to a medical device being utilized. While clinicians, and their support staff often closely monitor patients undergoing infusion or other medical therapies to prevent such unauthorized access, or unintended contamination, indeed, such unauthorized access and contamination does, unfortunately, occur, and the results of introducing unauthorized fluids (whether liquid or gas) through a VAD may result in infections or other serious medical conditions from developing and which may lead even to the death of the patient.

While attempts have been made by treating clinicians, healthcare workers, clinics and hospitals to educate both patients and other care givers regarding the dangers associated with tampering with VADs and related peripherals, such as PIC lines or catheters and PICC hubs, the problem has persisted. On the one hand, patients undergoing such therapies usually need some privacy while such therapies are conducted. However, this privacy often contributes to the undetected and unauthorized access and sometimes inadvertent contamination of these VAD assemblies. Moreover, in view of possible medical exigencies, clinicians must have ready access to both the infusion equipment, and the peripherals, in the event that emergency medical treatment must take place. In addition to the foregoing, and in light of the possible serious health consequences which may result from tampering with, or contamination of an infusion system, any inappropriately effected VAD warrants immediate attention, and intervention as soon as any tampering or contamination has been detected.

Consequently, those skilled in this art field have sought after a means for both inhibiting the unauthorized access to and contamination of VADs, and which further provides a means for quickly identifying when possible tampering and/or contamination has taken place, so that immediate medical intervention may be employed to prevent serious harm from happening to the patient undergoing therapy. Therefore, a means which achieves the objectives, noted above, is the subject matter of the present invention.

### SUMMARY OF THE INVENTION

A first aspect of the present invention relates to a container for enclosing a medical device, and which includes a flexible sheet which is defined by opposite first and second sides, and a peripheral edge, and wherein the flexible sheet is sized so that it may be received about, and substantially completely encloses a medical device; an adhesive layer is deposited on one of the first or second sides of the flexible sheet, and in a narrow zone which is oriented near the peripheral edge of the flexible sheet, and wherein the adhesive layer is employed to adhere to one side of the flexible sheet so as to maintain the flexible sheet in an orientation where the flexible sheet completely encloses the medical device; and an adhesive layer release sheet is provided, and which is located in covering relation relative to the adhesive layer when the flexible sheet is not enclosing the medical device.

Still another aspect of the present invention relates to a container for enclosing a medical device and which includes a flexible, and at least partially transparent sheet which is defined by a first surface; an opposite, second surface; and a peripheral edge, and wherein the flexible sheet is sized so that when the flexible sheet is folded, at least in part onto itself, the folded, flexible sheet forms a cavity which receives, and encloses a medical device; a zone of adhesive deposited in a region of the second surface of the flexible sheet and which is adjacent to the peripheral edge thereof, and wherein the zone of adhesive is effective in adhesively securing a first portion of the second surface of the flexible sheet, to a second portion of the second surface when the flexible sheet is folded so as to form the cavity and which encloses the medical device, and wherein the zone of adhesive is located along the entire length of the peripheral edge so that the resulting cavity is substantially inaccessible once the medical device is received within the cavity; and an adhesive release sheet which is located in covering relation relative to the zone of adhesive and which is removed prior to the folding of the flexible sheet to form the cavity.

These and other aspects and forms of the present invention will be discussed in greater detail hereinafter.

### BRIEF DESCRIPTION OF THE DRAWINGS

Preferred embodiments of the invention are described below with reference to the following accompanied drawings.

FIG. 1 is a perspective, environmental view of a portion of a typical infusion arrangement and which is employed to deliver a source of a fluid to a patient undergoing medical treatment.

FIG. 2 is a greatly enlarged, partial view of an infusion system utilizing Vascular Access Devices (VADs) which are coupled in fluid delivering relation relative to a line or tube which is inserted into a patient.

FIG. 3 is a graphical depiction of a PIC line or catheter which is coupled in fluid receiving relation relative to three PICC hubs which are coupled to same. The three PICC hubs, as illustrated, represent one type of medical device (VAD) which may be enclosed within the container for enclosing a medical device of the present invention.

FIG. 4 is a graphical depiction of a flexible sheet which forms a feature of the present invention, and wherein an adhesive layer release sheet is being removed from same to expose an adhesive layer which is located therebeneath.

FIG. 5 is a graphical depiction of one form of the present invention and which is now ready to enclose a medical device in a manner as seen in FIG. 4.

FIG. 6 is a plan view of still another form of the present invention and which displays a pull tab to assist in the opening of the container as described.

### DETAILED DESCRIPTION OF THE PREFERRED EMBODIMENTS

This disclosure of the invention is submitted in furtherance of the constitutional purposes of the U.S. Patent Laws “to promote the progress of science and useful arts” (Article 1, Section 8).

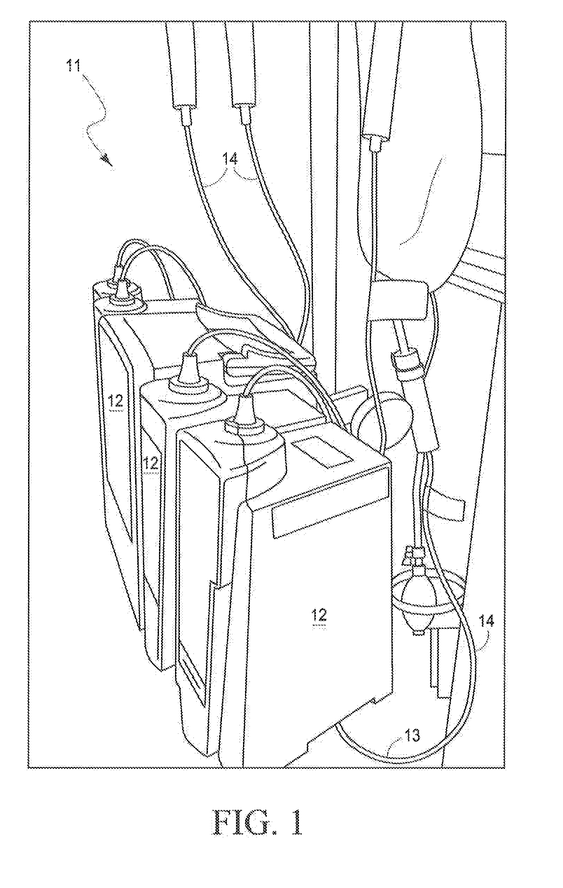

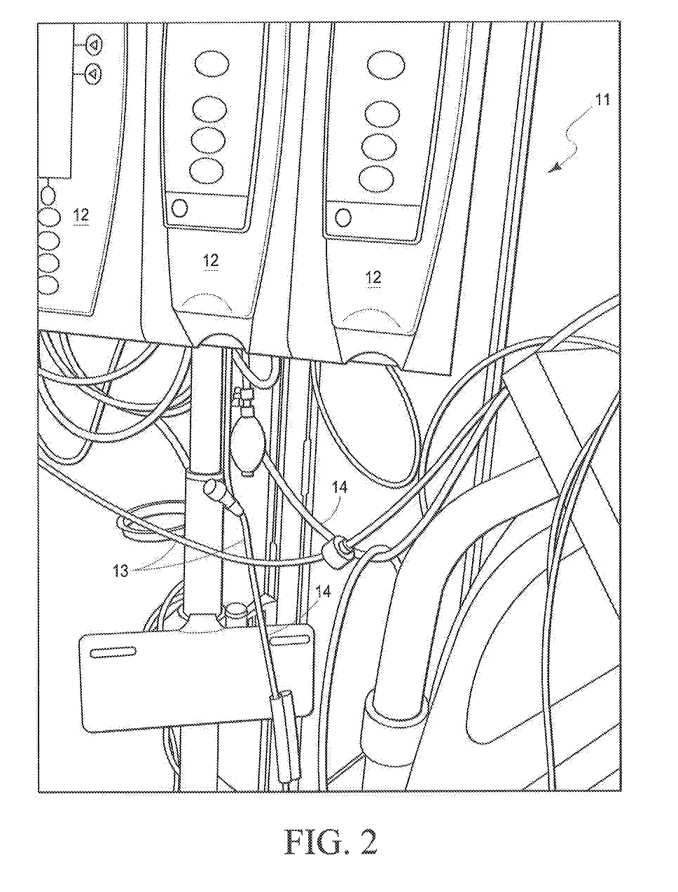

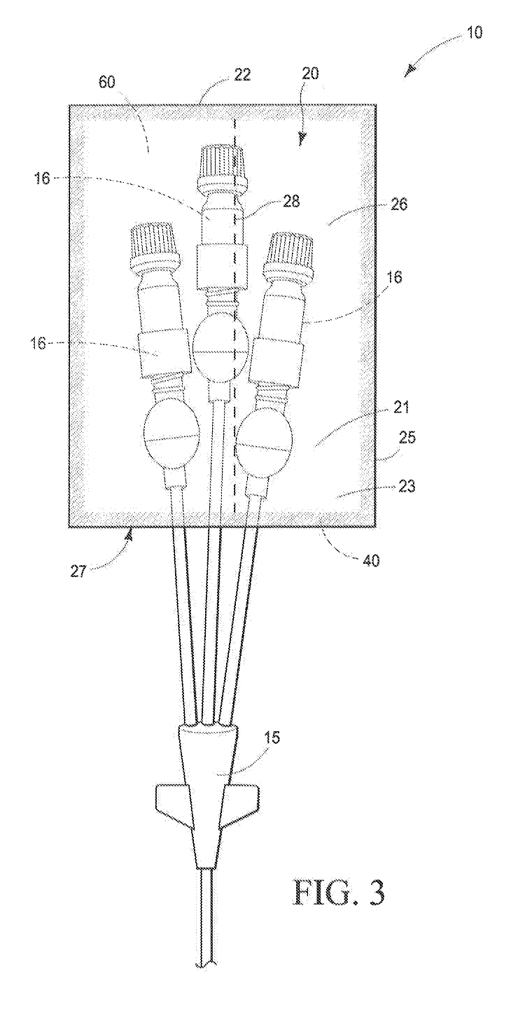

One possible form of the present invention is generally indicated by the numeral 10, and is best understood by a study of FIGS. 3, 4 and 5 respectively. As best understood by a study of the environmental view of FIG. 1, the present invention 10, as seen in FIG. 3, is useful when used for enclosing medical devices or peripheral features which are typically utilized in a general vascular infusion arrangement which is indicated by the numeral 11. These vascular infusion arrangements 11 are well known, and often include the use of several individual infusion pumps 12 which can supply various fluids, and other medicines, and which further can then be delivered in a vascular manner to a patient, not shown. The individual infusion pumps 12 (FIGS. 1 and 2) are coupled to an infusion given pathway which is generally indicated by the numeral 13. The infusion given pathway 13 often includes tubing 14 which is then coupled in fluid flowing relation relative to a line or tube, and which is then inserted into the patient. One type of a Vascular Access Device (VAD) is a PIC line or catheter 15 as seen in FIG. 3. In this type of VAD the PIC line 15 is coupled in fluid flowing relation relative to a multiplicity of PICC hubs 16 as seen in FIG. 3. The respective PICC hubs 16 are well known, and their operation does not need to be described in the present application. However, those skilled in the art will recognize that a multiplicity of PICC hubs or other VADs may be utilized based upon the type of medical treatment or regimen that the patient is currently undergoing.

As was discussed earlier in this patent application, as of late, treating clinicians, and other healthcare workers have witnessed or otherwise experienced an increase in the number of incidents where tampering or other inadvertent contamination of VADs has been detected. To inhibit the tampering that has sometimes been detected or reported, and also to prevent potential serious health problems from arising with the patient from ambient contamination, the present invention 10 has been conceived.

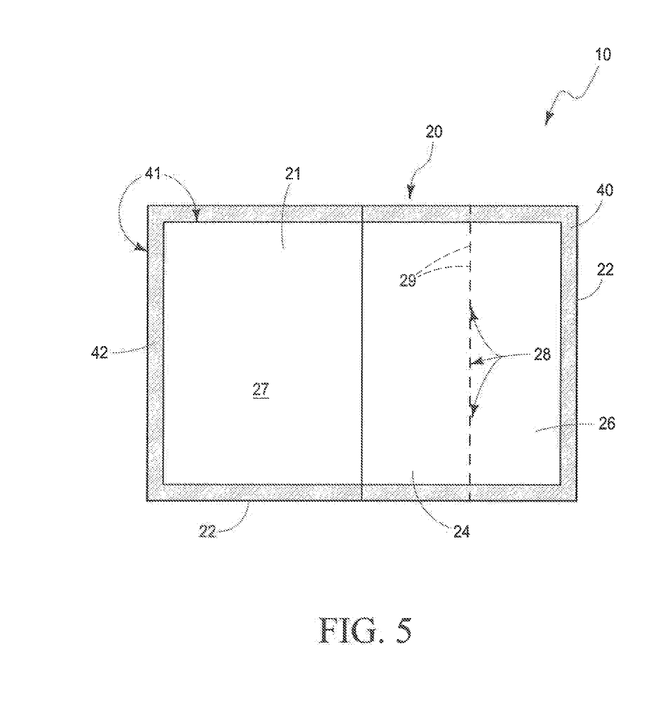

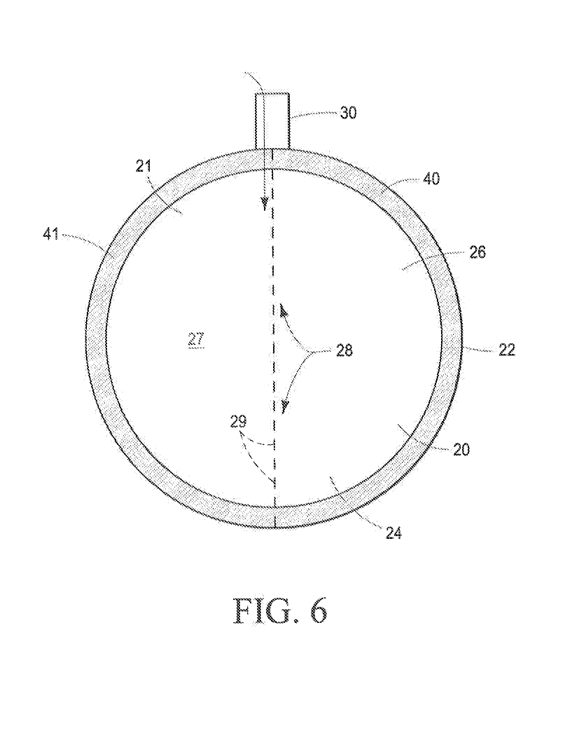

Referring now to FIGS. 3-5, one possible form of a container 10 for enclosing a medical device 16, such as a PICC hub 16, is seen in these drawings. In this regard, the container 10 for enclosing a medical device is formed of a flexible, fluid impervious sheet 20 which is typically fabricated from a synthetic polymer having a thickness dimension of typically less than about 1 mm. The flexible sheet 20 as seen in these drawings is depicted as having a square, or rectangular shape, but the main body 21 may have different shapes including circular (FIG. 6), oval and other irregular shapes depending upon the type, size, and shape of the medical device 16 which the flexible container 10 will enclose. The main body 21 of the flexible sheet 20 is at least, in part, transparent or translucent so that once the flexible container 10 is appropriately positioned or deployed to enclose a medical device 16, a clinician or caregiver, not shown, can continue to view the medical device in the event that the medical device malfunctions or some other situation or event occurs which requires immediate clinician intervention. The flexible sheet 20 is usually fabricated of a material which will permit a user to write on the exterior or first surface as will be described, below. This will permit a clinician, for example, to make appropriate notes regarding when the flexible container was put into place, and by whom. The flexible sheet 20 is defined, in part, by a peripheral edge 22. Still further, the flexible sheet 20 has a first surface or side 23, and an opposite or second surface or side 24. As illustrated in the drawings, and in one form of the invention, the flexible sheet 20 may be formed in a fashion where it has a preexisting or otherwise previously established medial fold line 25 which is formed therein. The medial fold line permits the flexible sheet to be easily folded, upon itself, in the manner which will be discussed, below. The medial fold line divides the second surface or side 24 into a first portion 26, and a second portion 27. This feature will also be discussed in greater detail in the paragraphs which follow. Still further the flexible sheet as seen in FIG. 5 or 6 typically includes a weakened region, or area, which is generally indicated by the numeral 28. The weakened region, as seen in the drawings is illustrated as being a scored line 29 which will allow a clinician or other attending and authorized healthcare worker to apply force in order to tear or otherwise rend open the container 10 when it is properly installed (FIG. 3) so as to gain access to the medical device 16, for appropriate medical purposes. In one form of the invention, this tearing or rending of the container 10 can be achieved by means of a pull tab 30 which is seen in FIG. 6. The pull tab 30, as illustrated, extends circumferentially, outwardly relative to the peripheral edge 22. By exerting a force on the pull tab 30 the scored line 29 separates to open the container 10. This feature makes the present invention particularly useful when a gloved healthcare worker needs to have rapid access to the medical device 16 which is enclosed within the container.

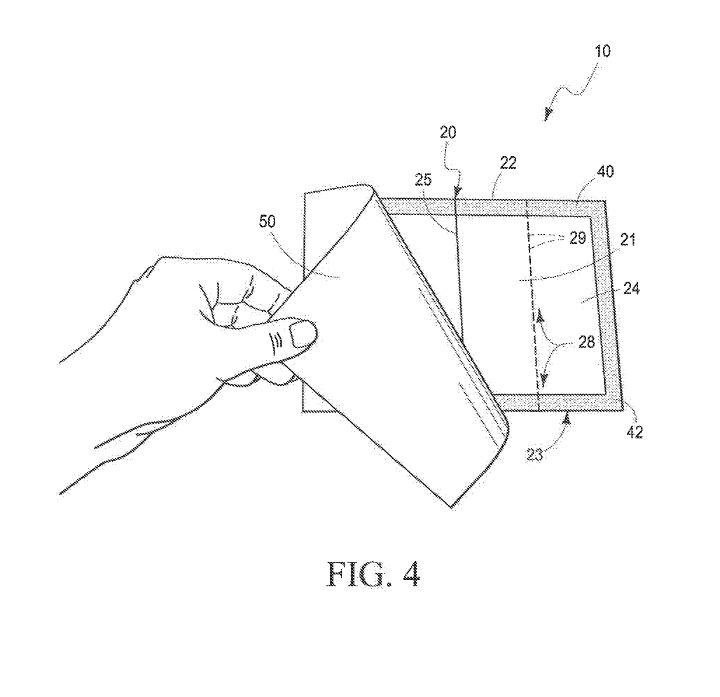

As seen in the drawings (FIGS. 5 and 6), the flexible sheet 20 includes an adhesive layer 40 which is applied to the second side or surface 24. The adhesive layer 40 is applied in a narrow adhesive zone 41 which is located adjacent to the peripheral edge 22. As depicted therein, the zone of adhesive 41 extends along the entire length of the peripheral edge 22. However, in some forms of the invention, the adhesive zone may extend along only a portion of the length of the peripheral edge 22. The adhesive layer 40 has a top surface 42 which when exposed, and placed in contact with either the main body 21 of the flexible sheet 20, or with another portion of the zone of adhesive, creates a very strong adhesive bond which typically cannot be separated from the flexible sheet. The adhesive strength is selected so that the amount of force that would be applied to open the container 10 causes a distortion or tears the flexible sheet 20. This adhesive is selected to be this strong so that any attempt to tamper with the medical device by applying force to the container 10 will cause such distortion to the flexible sheet that a clinician can easily visually detect the tampering attempt, and then can take immediate action. The top surface 42 of the adhesive layer 40 is initially covered with an adhesive layer release sheet 50, (FIG. 4) which covers the top surface 42 so that the flexible sheet 20 can be moved about in a clinical setting, prior to installation, without the flexible sheet adhering to other surfaces. When the flexible container 10 is installed to enclose the medical device 16 (FIG. 3), the adhesive layer release sheet 50 is first removed (FIG. 4) from the adhesive layer 40, and thereafter, the flexible sheet 20 is folded or otherwise received about and substantially completely encloses the medical device 16 (FIG. 3).

In another form of the invention, not shown, the container may take on the form of a rigid, transparent clam shell arrangement which can be folded onto itself to form an interior cavity 60 and which is sized so as to enclose the medical device 16 as seen in FIG. 3. In this other form of the invention the container 10 may be closed by means of the adhesive layer 40, as earlier described, or by other well-known means including by a lock which is made integral with the container.

### Operation

The operation of the described embodiment of the present invention is believed to be readily apparent, and is briefly summarized at this point. In its broadest aspect, the present invention relates to a container 10 for enclosing a medical device 16, and which is formed of a flexible sheet 20 which is defined by opposite first and second sides 23 and 24, respectively, and which has a peripheral edge 22. The flexible sheet 20 is sized so that it may be received about, and substantially completely encloses the medical device 16. The present invention further includes an adhesive layer 40 which is deposited on one of the first or second sides 23 and 24, respectively, of the flexible sheet 20, and in a narrow zone 41 which is oriented near the peripheral edge 22 of the flexible sheet 20. The adhesive layer 40 is employed to adhere to one side of the flexible sheet 20 so as to maintain the flexible sheet 20 in an orientation where the flexible sheet completely encloses the medical device 16. The present invention 10 further includes an adhesive layer release sheet 50 which is located in covering relation relative to the adhesive layer 40 when the flexible sheet 20 is not enclosing the medical device 16.

In one form of the invention as seen in the drawings, the container 10 is fabricated of a synthetic substrate, and wherein the flexible sheet 20 is at least partially transparent. Still further, the flexible sheet 20, in one form of the invention, may be weakened 28, so as to facilitate an opening of the container by an application of a predetermined amount of physical force. Still further, the weakened region 28 of the flexible sheet may be defined, in one form of the invention, by a scored line 29. In one form of the invention, as depicted, the adhesive layer 40 has an adhesive strength which resists separation from the flexible sheet 20 to such a degree that the flexible sheet 20 is visibly distorted when force is applied to open same. In the arrangement as seen in the drawings the flexible container 10 of the present invention is a single-use container. Still further, the flexible sheet 20 is substantially fluid impervious. As illustrated in the drawings the container encloses a medical device 16 which, in the form of the invention, as illustrated, permits selective vascular access to an individual undergoing a clinician's supervised medical treatment. As noted earlier, the container 10 may also be useful in inhibiting contamination of the medical device 16 from sources which come from the immediate ambient environment. Further, and as seen in the drawings, the flexible container 10, and the medical device 16 may include at least one VAD 16 which is coupled in fluid flowing relation relative to a catheter line or tube 15. As earlier discussed, the distortion and tearing of the flexible sheet 20 which forms the container 10 indicates a potential tampering with the medical device 16 which is enclosed within the flexible container 10.

More specifically, the present invention relates to a container 10 for enclosing a medical device 16, and which includes in one form, a flexible, and at least partially transparent sheet 20 which is defined by a first surface 23; an opposite second surface 24; and a peripheral edge 22, and wherein the flexible sheet 20 is sized so that when the flexible sheet 20 is folded, at least in part, onto itself the folded flexible sheet 20 forms a cavity 60 which receives an enclosed medical device 16. The container 10 includes a zone of adhesive 41 which is deposited in a region of the second surface 24 of the flexible sheet 20, and which is located adjacent to the peripheral edge 22 thereof. The zone of adhesive 41 is effective in adhesively securing a first portion 26 of the second surface 24 of the flexible sheet 20 to a second portion 27 of the second surface 24 when the flexible sheet 20 is folded, so as to form a cavity 60. The cavity 60 encloses the medical device 16. The zone of adhesive 41 is located along the entire length of the peripheral edge 22 so that the resulting cavity 60 is substantially inaccessible once the medical device is received within the cavity 60. An adhesive release sheet 50 is provided, and which is located in covering relation relative to the zone of adhesive 41, and which is removed prior to the folding of the flexible sheet to form the cavity 60 (FIG. 4).

Again, as was previously discussed, the flexible sheet is weakened 28, so as to facilitate an opening of the container 10 by an application of a predetermined amount of physical force. This force can be transmitted to the weakened region 28 by means of the pull tab 30 as seen in FIG. 6. Because of its single-use nature, clinicians can readily visually determine whether unauthorized individuals have attempted to tamper with the medical device 16 which is enclosed in the cavity 60.

Therefore, it will be seen that the present invention provides an easy and convenient means whereby a clinician can secure a medical device 16 against tampering or unauthorized access, and further provides a means whereby unintended contamination may be avoided. The present invention, when utilized properly, impedes possible blood infections or other health hazards, and can further prevent the unauthorized access to a medical device as described earlier in this application.

In compliance with the statute the invention has been described in language more or less specific as to structural and methodological features. It is to be understood, however, that the invention is not limited to the specific features shown and described since the means herein disclosed comprise the preferred forms of putting the invention into effect. The invention is therefore claimed in any of its forms or modifications within the proper scope of the appended Claims appropriately interpreted in accordance with the Doctrine of Equivalents.

We claim:

1. A container for enclosing a medical device, comprising:
a flexible sheet which is defined by opposite first and second sides, and a peripheral edge, and wherein the flexible sheet is sized so that it may be received about, and substantially completely encloses a medical device;
an adhesive layer deposited on one of the first or second sides of the flexible sheet, and in a narrow zone which is oriented near the peripheral edge of the flexible sheet, and wherein the adhesive layer is employed to adhere to one side of the flexible sheet so as to maintain the flexible sheet in an orientation where the flexible sheet completely encloses the medical device; and
an adhesive layer release sheet which is located in covering relation relative to the adhesive layer when the flexible sheet is not enclosing the medical device.

2. A container as claimed in claim 1, and wherein the flexible sheet is at least partially transparent.

3. A container as claimed in claim 1, and wherein a region of the flexible sheet is weakened so as to facilitate an opening of the container by an application of a predetermined amount of physical force.

4. A container as claimed in claim 3, and wherein the weakened region of the flexible sheet is defined by a scored line.

5. A container as claimed in claim 1, and wherein the adhesive layer has an adhesive strength which resists separation from the flexible sheet to a degree that the flexible sheet is visibly distorted.

6. A container as claimed in claim 1, and wherein the flexible container is a single-use container.

7. A container as claimed in claim 1, and wherein the flexible sheet is substantially fluid impervious.

8. A container as claimed in claim 1, and wherein the medical device is one which permits selective access to a vascular access device and which is fluid-flowingly coupled to an individual undergoing a clinician supervised medical treatment.

9. A container as claimed in claim 8, and wherein the medical device includes at least one vascular access device which is coupled in fluid flowing relation relative to a catheter line.

10. A container as claimed in claim 1, and wherein a distortion and tearing of the flexible sheet which forms the container indicates a potential tampering with the medical device which is enclosed within the flexible container.

11. A container for enclosing a medical device, comprising:
a flexible, and at least partially transparent sheet which is defined by a first surface; an opposite, second surface; and a peripheral edge, and wherein the flexible sheet is sized so that when the flexible sheet is folded at least in part onto itself, the folded, flexible sheet forms a cavity which receives, and encloses a medical device;
a zone of adhesive deposited in a region of the second surface of the flexible sheet and which is adjacent to the peripheral edge thereof, and wherein the zone of adhesive is effective in adhesively securing a first portion of the second surface of the flexible sheet, to a second portion of the second surface when the flexible sheet is folded so as to form the cavity and which encloses the medical device, and wherein the zone of adhesive is located along the entire length of the peripheral edge so that the resulting cavity is substantially inaccessible once the medical device is received within the cavity; and
an adhesive release sheet which is located in covering relation relative to the zone of adhesive and which is removed prior to the folding of the flexible sheet to form the cavity.

12. A container as claimed in claim 11, and wherein a region of the flexible sheet is weakened so as to facilitate an opening of the container by an application of a predetermined amount of physical force.

13. A container as claimed in claim 12, and wherein a distortion and tearing of the flexible sheet which forms the container indicates a potential tampering with the medical device which is enclosed within the flexible container.

14. A flexible container as claimed in claim 13, and wherein the medical device is one which permits selective vascular access to an individual undergoing clinician supervised medical treatment.

15. A flexible container as claimed in claim 14, and wherein the flexible container is a single-use container.

16. A container for enclosing a medical device, comprising:
a transparent enclosure having first and second portions which are hingedly mounted together and which, when moved into contact with each other defines an internal cavity which is sized so that it may enclose a medical device; and
a coupling means for securing the first and second portions together to prevent unauthorized access to the medical device which is enclosed in the internal cavity.

17. A container as claimed in claim 16, and wherein the transparent enclosure comprises a single-use folded, flexible sheet.

18. A container claimed in claim 16, and wherein the transparent enclosure takes the shape of a transparent semi-rigid clam shell.

19. A container as claimed in claim 16, and wherein the coupling means is an adhesive layer which joins the first and second portions together.

20. A container as claimed in claim 16, and wherein the coupling means releasably couples the first and second portions together.
